(19) United States
(12) Patent Application Publication
(10) Pub. No.: US 2014/0369886 A1
(43) Pub. Date: Dec. 18, 2014

(54) Water treatment

(71) Applicants: Joseph A. King, Wayzata, MN (US); John Hill, Plymouth, MN (US); Nicole Weulander, Prior Lake, MN (US)

(72) Inventors: Joseph A. King, Wayzata, MN (US); John Hill, Plymouth, MN (US); Nicole Weulander, Prior Lake, MN (US)

(21) Appl. No.: 14/120,934

(22) Filed: Jul. 11, 2014

Related U.S. Application Data
(63) Continuation-in-part of application No. 13/987,929, filed on Sep. 17, 2013.
Application No. 13/987,929 is a division of application No. 12/800,692, filed on May 20, 2010, now Pat. No. 8,591,748.
Application No. 12/800,692 is a continuation-in-part of application No. 12/315,285, filed on Dec. 2, 2008, now Pat. No. 7,875,191.
Application No. 12/315,285 is a continuation of application No. 11/999,654, filed on Dec. 6, 2007, now Pat. No. 7,501,067.
Application No. 11/999,654 is a division of application No. 10/928,668, filed on Aug. 26, 2004, now Pat. No. 7,347,934.
Application No. 10/928,668 is a continuation-in-part of application No. 13/986,763, filed on Jun. 4, 2013.
(60) Provisional application No. 61/959,331, filed on Aug. 21, 2013.
(60) Provisional application No. 61/126,105, filed on May 1, 2008.

(51) Int. Cl. A61L 2/238

(52) U.S. Cl. CPC A61L 2/238
USPC 422/25; 422/261

(57) ABSTRACT

An apparatus and a method of extending a use life of a body of recreational water while reducing a concentration of chlorine required to effectively sanitize the body of recreational water, the method comprising the steps of adding a sanitizing concentration of total available chlorine to the body of recreation water that is suitable for human partial immersion in the body of recreation water; adding a source of silver ions and a source of 1,3-Dichloro-5,5-dimethylhydantoin to the body of recreational water to lower the concentration of chlorine required to effectively sanitize the body of recreational water; and maintaining a concentration of silver ions and a concentration of the source of 1,3-Dichloro-5,5-dimethylhydantoin effective at extending the use life of the body of recreational by at least 2 folds before a required chlorine shocking of the recreational body of water.

### CROSS REFERENCE TO RELATED APPLICATIONS

This application claims priority to U.S. provisional application Ser. No. 61/959,331, filed on Aug. 21, 2013, titled Water Treatment and is a continuation-in-part application of pending U.S. application Ser. No. 13/987,929, filed on Sep. 17, 2013, titled Water Treatment, which is a divisional application of U.S. application Ser. No. 12/800,692, filed on May 20, 2010 titled Water Treatment (now U.S. Pat. No. 8,591,748), which is a continuation-in-part of U.S. application Ser. No. 12/315,285 filed Dec. 2, 2008 (now U.S. Pat. No. 7,875,191), which is a continuation of application Ser. No. 11/999,654 filed on Dec. 6, 2007, (now U.S. Pat. No. 7,501,067), which is a division of application Ser. No. 10/928,668 filed on Aug. 26, 2004, (now U.S. Pat. No. 7,347,934) and is also a continuation-in-part application of pending U.S. application Ser. No. 13/986,763, filed on Jun. 4, 2013, titled ION ENHANCEMENT, which claims priority to U.S. Provisional Application Ser. No. 61/126,105; filed on May 1, 2008; titled ION ENHANCEMENT.

### STATEMENT REGARDING FEDERALLY SPONSORED RESEARCH OR DEVELOPMENT

None

### REFERENCE TO A MICROFICHE APPENDIX

None

### BACKGROUND OF THE INVENTION

Contained bodies of water where a person immerses himself or herself partly or wholly in the body of water can be found in swimming pools, spas, hot tubs, jetted tubs as well other containers. Generally, the person immerses his or her body partially or wholly in the body of water in order to obtain the effects of the water. In order to make the body of water safe for body contact it is necessary to kill harmful microorganisms in the water such as bacteria which can be done by dispensing antibacterial materials into the water. These types of water characteristics can be referred to as water safety characteristics. In addition, from time to time other water characteristics of the body of water, such as water comfort characteristics are adjusted. For example, the water temperature may be changed or in some cases the pH of the water may be adjusted sometimes both for safety purposes and for the comfort of the water user. In general, a pump circulates the water in a container so as to provide a body of water with uniform water characteristics as well as to remove unwanted materials or debris from the water. During the circulation of the water dispensable materials such as minerals or other ion yielding materials can be controllable dispensed into the water through either inline dispensers or floating dispensers to control the microorganisms in the water. Other water characteristics such as a water temperature can be controlled by a heating or cooling the water as it flows through a heat transfer unit. In either case the water characteristics whether safety or comfort characteristics can be changed to make the water suitable and safe for the user. While water characteristics in the containers have been periodically monitored and controlled by adding dispensable material to the water a feature of the invention is that the water characteristics, such as water comfort characteristics and water safety characteristics, can be monitored and controlled on-the-go. This allows the water characteristics to be maintained within close ranges, which can make a user's recreational experience in the water not only more comfortable but also insure that the water does not contain harmful organisms. The invention described herein is well suited to use with small recreational water systems such as pools, spas, hot tubs or the like either as an after-market item in those systems have an existing circulation system it can be introduced during the manufacture of the pools, spas, hot tubs or the like. The concept of treating water with a source of metallic ions to kill bacteria in a body of water is known in the art. A metallic ion such as a silver ion is an effective sanitizer for a body of water including recreational water such as swimming pools, spas, jetted tubs or the like and is a preferred material because it is generally easier and safer to use than other known sanitizers. A further advantage of using silver ion as a sanitizer is that silver ion minimizes the need for pH adjustment of the body of water. However, if the concentration of metallic ions such as silver ions in a body of water is too low the ability to kill microorganisms is reduced or lost. On the other hand if the concentration of metallic ions such as silver ions is too high it can be harmful to those who use the body of water and/or potentially lead to undesired effects such as causing the user's skin to turn yellow and staining clothes. Thus when silver ion is used as a disinfectant in a body of water one generally wants to maintain the concentration of the silver ion in a range that is effective killing microorganisms without leading to the undesired effects associated with higher levels of silver ions.

Traditionally, the sources of metallic ions used to kill bacteria in recreational water have been limited to metallic ion donors that are readily soluble in the recreational water in order to maintain an effective concentration of the sanitizers in the body of water. Silver chloride (AgCl), for example, has been a commonly used bactericide for releasing silver ions into the body of water to effectively kill microorganisms. Sodium bromide has also been known to be used with silver chloride to provide an additional and alternative water disinfection system. One of the problems associated with the use of silver as a sanitizer for killing microorganisms is that silver has a tendency to complex with other compounds and become increasingly insoluble thereby reducing the effective microorganisms killing ability of the silver as a sanitizer. For example, it would not be anticipated that silver chloride when used in combination with sodium bromide would be an effective disinfectant system because of the combination's tendency to form insoluble bromide crystals, which are not believed to be biologically active in aqueous environments. However, it has been discovered that if silver forms a complex with hydantoins, the silver will remain soluble to a higher degree thereby retaining the silver's antimicrobial activity.

In general, bodies of recreational waters, such as swimming pools, spas and hot tubs, are cleaned and treated for microbiological contaminants before the bodies of recreational waters are in a sanitized and ready to use condition. Once in the sanitized and ready to use condition microbiological contaminants are killed or controlled in the bodies of recreational waters by maintaining a level of sanitizer in the bodies of recreational waters.

At a certain point over time, the maintenance sanitizing process for a body of recreational water ceases to be as effective in killing or controlling the growth of the microbiological contaminants in the body of recreational water. Causes for the aforementioned may include overloading bathers in the body of recreational water to a point that it is beyond the normal effectiveness of the sanitizer, significant changes to the pH of the body of recreational water, equipment failures, accumulation of organic waste in the body of recreational water, and possible mutation of the microbiological contaminants leading to a resistance to the maintenance level of the sanitizer in the bodies of recreational waters.

Once the maintenance sanitizing process for the body of recreational water ceases to be effective in killing or controlling the growth of microbiological contaminants the body of recreational water is no longer in the sanitized and ready to use condition and a use life of the body of recreational water ends. The use life of a body of recreational water may be defined as a period of time during which the body of recreational water is in the sanitized and ready to use condition.

The conventional way to return a body of recreational water back to the sanitized and ready to use condition and start a new use life for the body of recreational water is to “shock” the body of recreational water with a heavy dose of sanitizer that is suitable to kill all of the microbiological contaminants in the body of recreational water. The sanitizer used for shocking the body of recreational water may be different from the sanitizer that was used to maintain the body of recreational water in the sanitized and ready to use condition. Alternatively, the sanitizer used for shocking the body of recreational water may be the same as the sanitizer that was used to maintain the body of recreational water in the sanitized and ready to use condition but in a much heavier dosage.

It is recommended in the art that bodies of recreational water such as swimming pools, spas and hot tubs be regularly shocked at least once weekly or bi-weekly under normal use and normal condition to keep the bodies of recreational water safe and clean. Shocking should even be done more frequently if the bodies of recreational water are used heavily for extended time periods. In addition, for outdoor swimming pools, one should shock more frequently after large amounts of rain or during extended periods of hot, sunny weather.

Chlorine is a widely used sanitizer or disinfectant for the purpose of not only maintaining a body of recreational water in a sanitized and ready to use condition but also for the purpose of shocking the body of recreational water in order to bring the body of recreational water back to a sanitized and ready to use condition due to chlorine's highly effectiveness in killing and controlling microbiological contaminants. Although chlorine is an effective sanitizer, it is well known in the art that chlorine must be applied frequently in order to maintain chlorine's efficacy in the body of recreational water. In addition, chlorine readily forms various chloramines (monochloramine, dichloramine, and trichloramine), which are irritating to the user's eyes, nose and lung and leads to the “chlorine” smell.

When used to stabilize or maintain a level of sanitizer in a body of recreational water for human use the Florida Department of Health and the National Spa and Pool Institute (NSPI®), which is a watchdog organization for the spa and swimming pool industry, recommends a chlorine use level in the range of 2-3 ppm for swimming pools and a chlorine use level in the range of 3-5 ppm for spas. When shocking the body of recreational water to bring the body of recreational water back to the sanitized and ready to use condition, the level of chlorine in the body of recreational water may be brought up to a break point chlorination, typical in the range of 10-20 ppm chlorine, in order to not only provide for complete decontamination and effective disinfection but also to effectively remove the chloramines present in the body of recreational water. Once the shocking process is completed the level of chlorine in the body of recreational water is allowed to fall down to less than 5 ppm before the body of recreational water is back in the sanitized and ready to use condition.

One of the draw backs in using chlorine is that exposure to high level of chlorine, such as during the shocking stages of the body of recreational water, may lead to corrosion and damage to not only the surfaces and equipment of the body of recreational water but also damage to the area in the vicinity of the body of recreational water.

There thus is a need to prolong the use life of bodies of recreational waters that uses a chlorine-based shock and having a use life that is at least in part maintained by a chlorine-based sanitizer to reduce damage to the bodies of recreational waters and the equipment associated therewith by reducing the number of required shocking while still maintaining the effectiveness of the sanitizer in the bodies of recreational waters.

### SUMMARY OF THE INVENTION

Briefly, the present invention comprises a method and a device for extending a use life of a body of recreational water while reducing a concentration of chlorine required to effectively sanitize the body of recreational water with the method generally comprising the steps of adding a sanitizing concentration of total available chlorine to the body of recreation water that is suitable for human partial immersion in the body of recreation water, adding a source of silver ions and a source of 1,3-Dichloro-5,5-dimethylhydantoin to the body of recreational water to lower the concentration of chlorine required to effectively sanitize the body of recreational water, and maintaining a concentration of the source of silver ions and a concentration of the source of 1,3-Dichloro-5,5-dimethylhydantoin effective at extending the use life of the body of recreational by at least 2 folds before a required chlorine shocking of the recreational body of water.

The device generally comprises a dispenser for extending a use life of a body of recreational water sanitized at least in part by chlorine while reducing a concentration of chlorine required to effectively sanitize the body of recreational water, the dispenser including a first housing having a water accessible compartment containing a silver ion donor with the water accessible compartment of the first housing releasing a concentration of silver ions that lowers the concentration of chlorine required to effectively sanitize the body of recreational water when contacted by the body of recreational water containing a concentration of chlorine. The dispenser also includes a second housing having a water accessible compartment containing a source of 1,3-Dichloro-5,5-dimethylhydantoin with the water accessible compartment of the second housing releasing a concentration of 1,3-Dichloro-5,5-dimethylhydantoin effective at extending the use life of the body of recreational by at least 2 folds before a required chlorine shocking of the body of recreational water when contacted by the body of recreational water containing the concentration of chlorine and the concentration of silver ions. For example, it has been discovered that the above method and device enables bodies of recreational water, which normally require at least weekly shocking under normal use and normal condition maybe reduced to a required shocking of once every 4 weeks or once every 8 weeks or beyond while still keeping the bodies of recreational water in a safe and clean condition.

### BRIEF DESCRIPTION OF THE DRAWINGS

FIG. 1 shows a graph of the measured dissolved silver concentrations each week for the duration of a Spa Study 1;

FIG. 2 shows a graph of the presentation of dissolved silver concentrations each week for the duration of a Spa Study 2 as compared to the theoretical calculations;

FIG. 3 shows a graph of is the presentation of dissolved silver concentrations each week for the duration of a Spa Study 3 as compared to the theoretical calculations;

FIG. 4 is a table showing the effect that the bathers have on the spa water of Spa Study 3.

FIG. 5 shows a dispenser having a housing with a compartment containing a source of 1,3-Dichloro-5,5-dimethylhydantoin and a silver ion donor comprising silver chloride therein; and

FIG. 6 shows a dispenser having a first housing containing a source of 1,3-Dichloro-5,5-dimethylhydantoin and a second housing containing silver ion donor comprising silver chloride therein.

### DESCRIPTION OF THE PREFERRED EMBODIMENT

In order to maintain a body of water such as a pool, spa or hot tub in a sanitized and ready to use condition when using only a halogen sanitizer and a single source of heavy metal ions such as silver ions one needs to maintain a minimum level of halogen in the body of water. For example if chlorine is used in conjunction with a source of silver ions it is recommend that the minimum level of chlorine should be 1 ppm. Similarly, if bromine is used in conjunction with a source of silver ions the minimum level of bromine needed to maintain the body of water at a safe level is 2 ppm. With the above level of sanitizers the body of water becomes safe for human contact. A recommended minimum halogen level for the body of water may vary depending on the tests standards, for example, one known EPA test standard requires the water have no more than 200 cfu of heterotrophic bacteria per ml; no more than one cfu of Enterococcus sp. Per 50 ml and no more than one cfu of Escherichia coli per 50 ml. The above levels of bacteria are generally consider safe levels of bacteria for bodies of water such as pools and spas and have been approved as such by regulatory governmental bodies such as the EPA. Another test standard requires that the minimum halogen level be such that (a) an organism count in the recreational body of water of less than 200 organisms per milliliter; (b) a coliform bacteria count in the recreational body of water of less than 2 bacteria per 100 milliliters; and (c) an entercoccal organism count in the recreational body of water of less than 2 entercoccal organisms per 50 milliliters. To meet the above EPA test standards for the allowable number of harmful organisms in a body of water the minimum recommended halogen level of bromine is 2 ppm or greater and the minimum recommended halogen level of chlorine is 1 ppm or greater if the halogen is used in conjunction with a source of metal ions such as silver ions. Typically, the silver ions may be delivered through a dispenser that contains silver chloride. One may limit the water contacting the source of silver ions by limiting the size of the port area for water to come into contact with the silver chloride located in the dispenser. If additional sanitizers are used with the chlorine and the source of metal ions the EPA may recommend that the level of chlorine may be as low as 0.5 ppm. While the test standards may vary in each instance the body of water, which comes into external human contact must be sanitized to kill harmful organisms. The use of a source of silver ions in conjunction with a halogen and a compound containing a hydantoin ring provides a method and composition that kills harmful organisms with reduced levels of halogens without the need to use additional sanitizers. In one example a chlorine generator, when used with a source of silver ions and a compound containing a hydantoin ring, is set to maintain the level of chlorine in the pool or spa as low as 0.25 ppm, which is as much as 50% to 75% less than the minimum recommended halogen levels to maintain acceptable levels of bacteria in a body of recreational water such as a pool or spa when chlorine and a source of silver ions are used as the sanitizers. Typically, the chlorine generator is set to maintain a minimum chlorine level in the body of water at 1 ppm when used with a dispenser that includes a source of silver ions. Similarly, when a halogen generator, such as a bromine generator is used the bromine generator is set to maintain the level of bromine at 2 ppm when used with a dispenser that includes a source of silver ions. Typically, a conventional sanitizing system may use only a halogen and a single source of metal ions for microorganism control in a body of water. If the halogen is chlorine, the chlorine level is maintained at a minimum of 1 ppm. If the halogen is bromine the bromine level is generally maintained at a minimum of 2 ppm. If additional sanitizers are used such as multiple sources of metal ions one may be able to reduce the level of halogen such as chlorine in the body of water to 0.5 ppm. In the system described herein one can obtain microorganism control in a body of water by adding a compound containing a hydantoin ring to the body of water containing a halogen and a source of silver ions regardless of whether the compound containing a hydantoin ring has any biocidal properties. For example, if the halogen is chlorine one can obtain microorganism control in a body of water a with a chlorine level as low as 0.25 ppm when the source of metal ions is used in conjunction with a compound containing a hydantoin ring Thus, in the invention described herein the halogen generator can be set to maintain the halogen level in the pool or spa at 50% to 75% less than would be normally required to safely control the bacteria level in the pool or spa when a compound containing a hydantoin ring such as DMH is also used. For example, a chlorine generator can be set to maintain a minimum level of chlorine of 0.25 ppm in the body of water. The chlorine generator can be set to maintain the chlorine level in the body of water between 0.25 ppm and 1 ppm and the dispenser can include a source of silver ions and a compound containing a hydantoin ring such as DMH. In one example, a cartridge contains a source of silver ions, such as silver chloride, which may be coated onto a carrier using a binder and a hydantoin such as DMH. To determine if the level of bacteria in the water was acceptable with 0.5 ppm chlorine tests were carried out based on the EPA directive DIS/TSS-12 using over 144 water samples, which were examined for the levels of microorganisms in the water samples. To meet the EPA test requirements for pools and spas at least 85% of the samples had to have no more than 200 cfu of heterotrophic bacteria per ml; no more than one cfu of Enterococcus sp. Per 50 ml and no more than one cfu of Escherichia coli per 50 ml. The pool studies were carried out at temperatures ranging from 80.degree. F. to 90.degree. F. and the spa studies were carried out at temperatures ranging from 100.degree. F. to 104.degree. F. It was found that a chlorine concentration as low as 0.25 ppm, when used with a source of silver ions and a compound containing a hydantoin ring met the EPA standards for maintaining safe levels of bacterial in the body of water. Thus the concentration of chorine in the body of water is 50% to 75% less than normally required to safely control the bacteria level in the pool or spa. While a halogen generator such as a chlorine generator can be used in conjunction with the source of metals ions such as silver ions and a compound containing a hydantoin ring such as DMH one can also use a halogen generator such as a bromine generator with a source of metals ions such as silver ions and DMH to similarly lower the level of bromine in the body of water. Thus the invention encompass a method of maintaining a body of recreational water in a sanitized condition with use of a halogen and a source of silver ions wherein the halogen is maintained at less than 0.5 ppm if the halogen is chlorine and less than 2 ppm if the halogen is bromine by adding a compound containing a hydantoin ring to the recreational body of water. The level of halogen in the body of recreational water can be maintained with a halogen generator and the sanitized condition may have any or all of the following conditions: (a) an organism count in a recreational body of water of less than 200 bacteria per milliliter; (b) a number of coliform bacteria in the recreational body of water less than 2 organisms per 100 milliliter; or (c) a number of entercoccal organisms in the recreational body of water less than 4 organisms per 100 milliliter. Thus the sanitizing of the body of body of recreational water such as a pool or spa using a halogen in combination with a metal ion source and a compound containing a hydantoin ring the level of halogen in the body of water can be as much as 50% to 75% less than is normally minimum required to maintain the bacteria levels in the body of water at acceptable levels when only a halogen and a source of silver ions are used to control the bacteria. The compound containing a hydantoin ring may be either a halogenated compound or an unhalogenated compound. In one example a chlorine generator, when used with a source of silver ions and a compound containing a hydantoin ring, can be set to maintain the level of chlorine in the pool or spa between 0.25 ppm and 0.5 ppm, which is as much as 50% to 75% less than normally required to maintain acceptable levels of bacteria in the pool or spa when only chlorine is used with the source of silver ions.

Hydantoin structures are known complexing agents in silver-plating processes (R. J. Morrissey, U.S. Patent Application Publication no. 2005/0183961). Studies performed by the inventor have demonstrated that unhalogenated hydantoins, such as 5,5-dimethylhydantoin (DMH), tend to increase levels of dissolved silver. Studies performed by the inventor have also demonstrated the halogenated hydantoin such as Bromochlorodimethylhydantoin (BCDMH) also tends to increase levels of dissolved silver. While not fully understood it is believed that the aforementioned increased in solubility is due to the soluble complex between silver and hydantoin ring structures as it has been found the silver remains soluble to a higher degree than expected.

The present invention has found that compounds having a hydantoin ring including unhalogenated hydantoins, such as 5,5-dimethylhydantoin (DMH), has the qualities to interact with metal ion donors such as silver chloride or silver bromide to increase the solubility of the silver chloride or silver bromide in a water environment and aid in the disinfection process is not limited to just the halogenated hydantoin BCDMH alone but may include a broader category of N-halohydantoin compounds. For example, the inventor has discovered that in addition to BCDMH, the N-halohydantoin compound Dichlorodimethylhydatoin (DCDMH), which has been used commercially in household automatic toilet bowl cleaners and urinals, may also properly interact with silver from sources such as silver chloride or silver bromide in a body of recreational water such as spas, jetted tubs, swimming pools or the like to form a soluble complex to enhance the effectiveness of the silver in killing or controlling microorganisms in the body of recreational water.

In order to verify the above, the following test was performed using silver chloride as the donor of metal ions to demonstrate the enhancement of a silver concentration in a body of water when other types of N-halohydantoin compounds such as DCDMH is used in combination.

In the test, a 450-gallon Marquis® brand spa was used in performing 3 studies to evaluate the potential use of DCDMH to increase silver solubility in the presence of alternative disinfection systems such as sodium bromide. The spa comprised a dimensioned of 90″×90″×35.5″ with a water depth of approximately 25″ without bathers. The spa featured 43 jets and two pleated filter cartridges (Unicel 5CH-502), each having a filtration area of 50 square feet. Spa water was maintained between 100° F. (37.8° C.) to 104° F. (40° C.) and was circulated at least 2 hours daily.

In all three studies, the Dichlorodimethylhydantoin (DCDMH, CAS No. 118-52-5) was obtained from two sources, namely Aldrich® and Lonza, Inc. located in Fair Lawn, N.J. The DCDMH obtained from Aldrich® comprised a fine powder material consisting of 1,3-Dichloro-5, 5 dimethylhydantoin with a 98% purity. The Lonza DCDMH (Dantochlor®) comprised a combination of 80-83% 1,3-Dichloro-5,5dimethylhydation, 16-17% 1,3-Dichloro-5-ethyl-5-methylhydatoin, 0-2% monochloro-5-methylhydation. The DCDMH was introduced into the spa via spa cartridges, which were fabricated by adding approximately 75-100 grams of DCDMH or Dantochlor to an empty Spa Frog® BCDMH cartridge.

The tests revealed that one may use DCDMH alone or DCDMH in combination with other hydantoins such as found in (Dantochlor®), which in addition to the DCDMH, contains 1,3-Dichloro-5,5dimethylhydantoin, 16-17% and 1,3-Dichloro-5-ethyl-5-methylhydantoin and 0-2% monochloro-5-methylhydantoin to obtain the benefits of the invention described herein. That is, it has been found that DCDMH alone or in combination with other hydantoins provide a method of maintaining spa water suitable for human immersion during a period of at least two months without having to shock the spa water to remove chloramines. More specifically, the invention of reducing the period between shock events to a period of two months or more includes the steps of adding chlorine to the spa water; placing a source of silver ions in the spa water to lower a sanitizing concentration of chlorine required to effectively sanitize the spa water; and subjecting the spa water to bather use while maintaining the sanitizing concentration of chlorine between 0.25 and 1.5 ppm by adding either a hydantoin such as 1,3-Dichloro-5,5-dimethylhydantoin to the spa water or by adding a combination of hydantoins (such as in Dantochlor®) wherein the main hydantoin comprises 81.1% 1,3-Dichloro-5,5-dimethylhydantoin, with lesser amounts of other hydantoins such 16.1% of 1,3-Dichloro-5,5dimethylhydantoin, with the balance comprising 1,3-Dichloro-5-ethyl-5-methylhydantoin, and monochloro-5-methylhydantoin to the body of water. Although Dantochlor® has been mentioned as a commercially available source of DCDMH other sources of DCDMH which contain other hydantoins may also be used without departing from the spirit and scope of the invention.

The source of silver ions was obtained from a King Technology Inc. Spa Frog® Mineral Cartridge, which was randomly selected from King Technology Inc.'s production inventories for use in these studies and installed into an in-line system on the spa. These mineral cartridges release silver ions into the spa in the form of silver chloride. A different cartridge was used in each of the three studies.

During all three studies, the test spa was filled with fresh water prior to the initiation of each study of the three spa studies and the water balanced according to Taylor Technologies Pool & Spa Water Chemistry Manual. The pH of the water was reduced by the addition of sodium bisulfate (pH Down Balancer, GLB, Alpharetta, Ga.) to a range between 7.2 and 8.0. In Studies 2 and 3, a cartridge containing the DCDMH was then installed into the In-Line Frog System of the test spa at the same time that the Spa Frog Mineral Cartridge (silver source) was installed into the In-Line Frog System of the test spa. In Study 1, a Spa Frog® Mineral Cartridge (silver source) was installed into the In-Line Frog System of the test spa. A cartridge containing the DCDMH was installed into the In-Line Frog System of the test spa three weeks after the start of the testing period.

In Spa Study 1, water samples were taken and tested for a ten-week period. In Spa Study 2, water samples were taken and tested for a seventeen-week period. And for Spa Study 3, water samples were taken and tested for a seven-week period. It is noted that in Spa Study 3, bathers were also introduced to the spa water three weeks after the start of the testing period to test the affect that bathers had on the spa water.

The Spa Frog® Mineral Cartridge provide silver ions in the form of solid silver chloride (AgCl) distributed over a porous matrix. Water flowing through the matrix comes into contact with the AgCl resulting in the release of soluble silver ions to water. DCDMH is also released to water resulting in the formation of free chlorine and hydantoin structures. It would be anticipated that soluble silver ions would be depleted from spa water through the formation of silver chloride, an insoluble salt. However, during each of the three spa studies the actual silver concentration was higher than the calculated theoretical silver concentration. This is due to the formation of a novel silver-hydantoin complex, which we previously described.

### Water Testing

Chemical tests were performed with water samples obtained from each of the three spa studies for the chlorine concentration and also, the dissolved silver concentration. Additionally, the spa water's total alkalinity, turbidity, and pH were also tested and maintained within ranges accepted by the industry. The ideal pH for a spa is 7.20 to 7.60, however wider ranges are acceptable. In the studies, the average pH for Spa Study 1 was 7.31, Spa Study 2 showed an average pH of 7.27, and Spa Study 3 had an average pH of 7.37, which were all within the low end of the ideal pH for a spa.

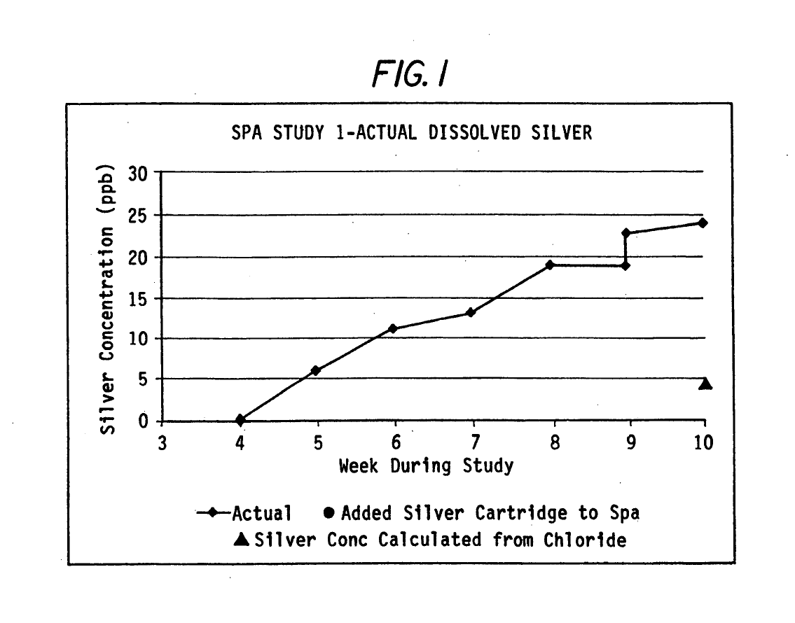

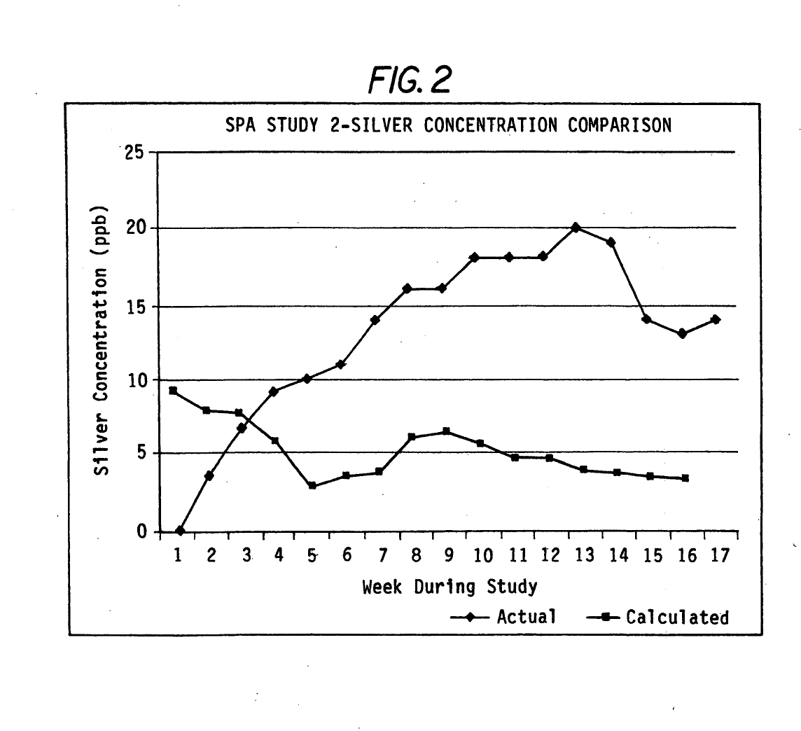

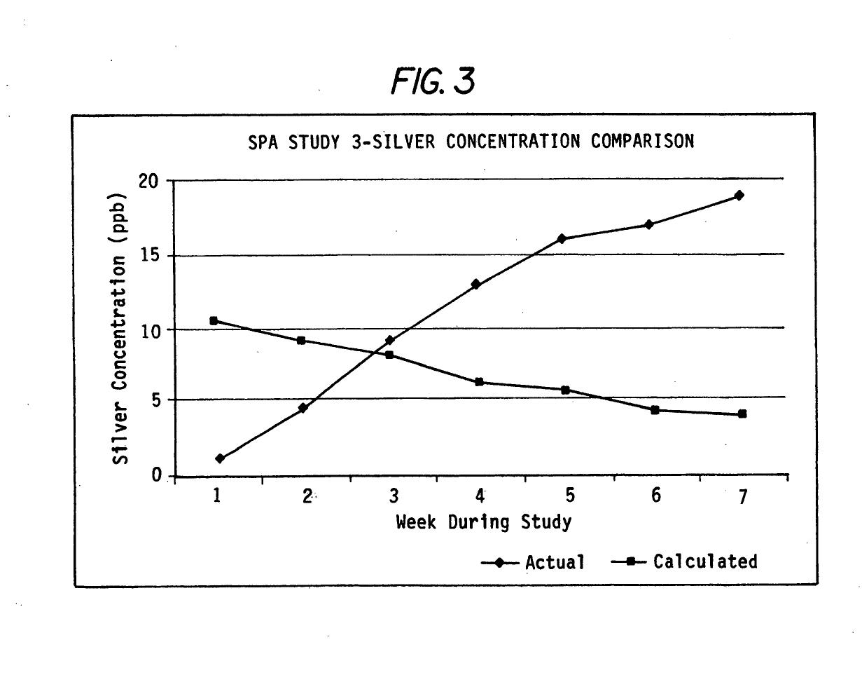

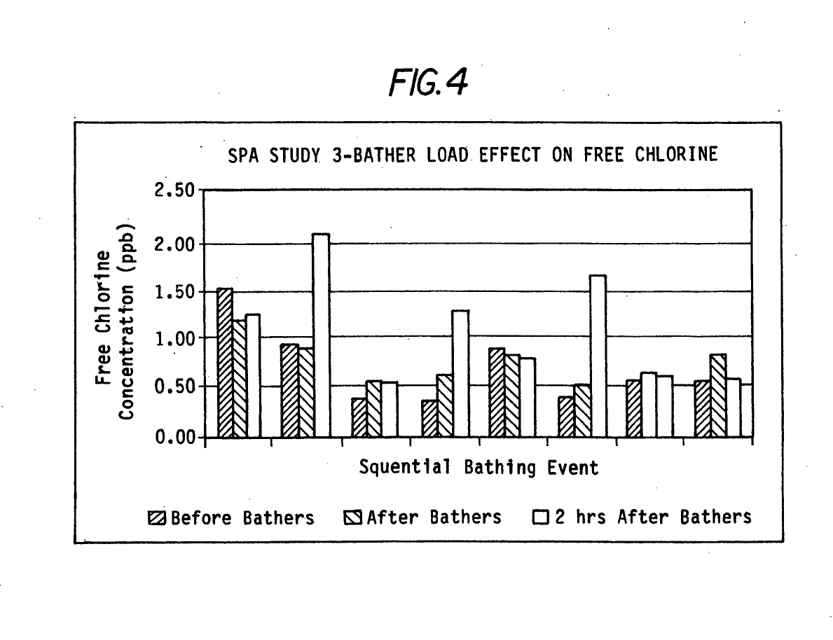

Result of the test for dissolved silver concentration are shown in FIG. 1 for Spa Study 1, are shown in FIG. 2 for Spa Study 2, and are shown in FIG. 3 for Spa Study 3. Chloride was tested during Spa Studies 2 and Spa Study 3 to provide a means to calculate the theoretical silver concentration based on the solubility product of silver chloride. FIG. 4 shows the effect that the bathers had on the spa water of Spa Study 3.

Free chlorine was measured to assess oxidizing potential for disinfection. The average levels of free chlorine in Spa Studies 1, 2, and 3 were 0.52 ppm, 0.68 and 0.79 ppm. Control of free chlorine concentrations in the observed range has not been previously possible when a solid source of chlorine has been dispensed from a simple cartridge device. It should be noted that although the aforementioned low levels of chlorine may be inadequate when DCDMH is used alone, the low levels of chlorine may be ideal for a combined used with Spa Frog Minerals. Therefore, DCDMH may be considered as an effective candidate for use with minerals in spas.

Total chlorine was measured to assess all forms of chlorine containing species present in spa water, some of which do not participate in the disinfecting process. The average total chlorine concentration for Spa Study 1 was 3.45 ppm (0.10 to 6.90 ppm range), Spa Study 2 averaged 6.16 ppm (range 0.12 to 14.4 ppm), and Spa Study 3 averaged 8.17 (range 0.17 to 15.8).

DCDMH's higher than expected concentrations of total chlorine may be contributed to the structure in that DCDMH has two chlorine atoms attached to a hydantoin ring. Additionally, it is believed that only one chlorine atom detaches from the ring, while the second may remain bonded. The hydantoin backbone with the one chlorine atom attached may possibly interact with the DPD reagent used to test for the total chorine resulting in higher total chlorine reading than what really is present.

Furthermore, the high total chlorine can be utilized as a chlorine bank, when there is a high demand. That is, it is reasonable to propose that the last chlorine atom detaches itself from the hydantoin ring with higher demand for use in the disinfection process such as in the presence of high bather load demand. Also, a decrease in total chlorine concentration has been observed after the bathers exit the test spa. Moreover, when the chlorine cartridge is empty the chlorine bank begins to fall and can be used as an indication that the cartridge needs to be replaced. Typically one DCDMH cartridge filled with 100 grams of DCDMH will last about 3-4 weeks depending on spa use. In view of the aforementioned, the total chlorine level may be monitored in the spa water to determine the quantity of chlorine that remains in the cartridge while the free chlorine level may be monitored in the spa water to determine disinfection potential.

FIG. 1 shows a graph of the measured dissolved silver concentrations each week for the duration of the Spa Study 1. The average dissolved silver concentration for Spa Study 1 was 16 ppb. During week 10 the chloride measured 160 ppm. The level of silver that would be anticipated based on theoretical calculations of the chloride would be about 4.2 ppb, however, the actual measured silver was 23 ppb. This is almost a 6-fold greater than would be anticipated.

FIG. 2 shows a graph of the presentation of dissolved silver concentrations each week for the duration of the Spa Study 2 as compared to the theoretical calculations based on the chloride measurement. The average dissolved silver concentration for Spa Study 2 was 13 ppb. By the end of Spa Study 2 the measured level of silver was at least 3-fold greater than would be anticipated based on theoretical calculations.

FIG. 3 shows a graph of the presentation of dissolved silver concentrations each week for the duration of the Spa Study 3 as compared to the theoretical calculations based on the chloride measurement. The average dissolved silver concentration for Spa Study 3 was 11 ppb. By the end of Spa Study 3 the measured level of silver was at least 5-fold greater than would be anticipated based on theoretical calculations. It appears from Spa Study 3 that bathers do not have an effect on dissolved silver concentrations. It is believed that Spa Study 3 had the lowest average silver concentration because the Spa Study 3 was run for seven (7) weeks compared to the testing duration of twelve (12) and eight (18) weeks for Spa Study 1 and 2, respectively. It is anticipated that if Spa Study 3 had been tested longer in duration the average dissolved silver concentration would have mostly likely been higher.

The above results of Spa Studies 1, 2, and 3, as shown in FIGS. 1, 2, and 3 thus supports the finding that the combination of other types of N-halohydantoin compounds such as DCDMH with a metal ion donor such silver chloride enhances a concentration of metal ions in a body of water by retaining or increasing the solubility of metal ions from other metal ion donors to retain the antimicrobial activity of the metal ions in the body of water.

Per the inventor's above findings, it is anticipated that N-halohydantoin compounds of the formula shown below can be used in this invention.

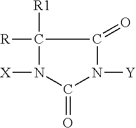

### Where

X is either H, Cl, or Br;
Y is either H, Cl, or Br;
R is an Alkyl group; and
R1 is an Alkyl group.

R and R1 are independently selected from alkyl groups (having from 1 to a plurality of carbons), and X and Y are independently selected from bromine, chlorine and hydrogen. In further regards to the above, as evidenced by the Inventor's use of the Lonza DCDMH (Dantochlor®), which comprised a combination of 1,3-Dichloro-5, 5 dimethylhydantoin, 1,3-Dichloro-5-ethyl-5-methylhydatoin, and monochloro-5methylhydation, a mixture of the derivatives of the above N-halohydantoin compounds can also be used.

FIG. 4 is a table showing the free chlorine concentration before and after two bathers used the spa for thirty (30) minutes increments on sequential days. The first columns correspond to the free chlorine level prior to the bathers entering the spa. The second columns represent the free chlorine level after the bathers exited the spa, and the third columns show the free chlorine concentration two hours after the bathers have exited the spa. Typically the next day after each bathing event the free chlorine stabilized between 0.5 and 1.0 ppm free chlorine even if 2 hours after spa use the free chlorine measured above 1.0 ppm. FIG. 4 also shows that when the free chlorine level is below 0.5 ppm, and bathers used the spa, the free chlorine goes up, instead of down. This can be attributed to the above-discussed chlorine-hydantoin bank, because as the demand for free chlorine goes up, the hydantoin releases the second chlorine on the ring to add to disinfection. Also the additional circulation from the jets of the spa and/or increases in water temperature may cause more DCDMH to dissolve into the spa water, and possibly increase the kinetics of the reaction.

The above results of Spa Studies 1,2, and 3 also indicates that unlike other sources of chlorine that have been previously evaluated by the inventor: (1) spa water chlorine concentrations can be controlled when DCDMH is dispensed from the existing Spa Frog® Bromine Cartridge; (2) at cartridge setting 2 in a 450-gallon spa, chlorine concentrations can be maintained at levels of 0.5 to 1.0 ppm and higher as needed; (3) concentrations of actual silver were 3 to 6-fold higher in spa water than would be anticipated by theoretical calculations based on silver chloride solubility; (4) due to the unique chemistry of DCDMH, total chlorine concentrations behave as a chlorine bank that is readily available under conditions of high chlorine demand, but without the risk of over chlorination; (5) spa water treated with DCDMH is as clear as, if not clearer, that water treated with BCDMH; and (6) after spa water has been balanced according to the saturation index, pH remains in a more neutral range (pH 7.4) as compared to spa water treated with BCDMH.

The present invention has also discovered that compounds having a hydantoin ring, and specifically Dichlorodimethylhydantoin (DCDMH) for example, when maintained at concentrations described herein, has the qualities to interact with metal ion donors such as silver chloride and a halogen such as chlorine in a body of recreational water to reduce a concentration of the chlorine required to effectively sanitize the body of recreational water that is that is suitable for human partial immersion in the body of recreation water while extending a use life of the body of recreational water by at least 2 folds and preferably at least 4 fold before a required chlorine shocking of the recreational body of water to return the body of recreational water back to a sanitized and ready to use condition and start a new use life for the body of recreational water.

For example, the art of recreational water recommends that bodies of recreational water such as swimming pools, spas and hot tubs be regularly shocked at least once weekly or bi-weekly under normal use and normal condition to keep the bodies of recreational water safe and clean. Shocking should even be done more frequently if the bodies of recreational water are used heavily for extended time periods. In addition, for outdoor swimming pools, one should shock more frequently after large amounts of rain or during extended periods of hot, sunny weather. It has been discovered that when Dichlorodimethylhydantoin (DCDMH), for example, is used with metal ion donors such as silver chloride and a halogen such as chlorine in a body of recreational water enables the body of recreational water, which normally require at least weekly shocking under normal use and normal condition maybe reduced to a required shocking of once every week to once every two months or beyond while still maintaining the body of recreational water in a safe and clean condition as described herein.

In a four months study performed by the Applicant approximately 100 grams of DCDMH was initially charged to each spa, 300 and 550 gallons to obtain a concentration of 0.333 and 0.182 g/gal, or 0.088 and 0.048 g/L respectively. Additional DCDMH was added to each spa during the study to maintain the concentration of DCDMH between 0.182 g/gal and 0.333 g/gal, which was done by placing sufficient DCDMH in a cartridge so that DCDMH was available for an extended period of time or by periodically topping off the DCDMH cartridge. A free floating Spa Frog® Mineral Cartridge dispenser randomly selected from King Technology Inc.'s production inventories was placed in the spa to provide the spa with a source of silver ions in the form of silver chloride.

To simulate real conditions volunteered bathers were provided with clean swimsuits, robes, sandals and towels, and each was required to take a soapy shower before entering one or the other spa. The bathers' use of the spas alternated between four and five use days per week. The 300 gallon spa was loaded with two bathers, and 550 gallon spa was loaded with four bathers, and each bather used the relevant spa for a total of 30 minutes with each use consisting consisted of two 15-minute intervals in the spa, separated by a 7 to 15 minutes for bather safety as it is not recommended that a spa user remain in the spa for more than 15 minutes at one time.

The data maintenance testing suggests that the Total Heterotrophic plate counts were controlled the following day, without shocking. It was the decision of the Study Director to shock the spas after the failing plates were recorded. The method for Total Heterotrophic requires that plates be examine 48 hours after incubation, so there is a 2-day lag. Results of the testing also revealed a correlation between the amount of DCDMH in the body of water and the frequency of shocking in that the 4 month study resulted in both spas only requiring to be shock twice each, which is far less than the art recommended weekly or bi-weekly shocking under normal use and normal conditions without DCDMH.

Based on the test results it also appears that the amount of organic material, which is normally removed by periodic shocking with chlorine, was also substantially removed on an ongoing basis using the available chlorine from the DCDMH molecule. In addition it also does appear that the left over DMH in the water functions to stabilize the available chlorine atoms in the water and prevent the formation of hypochlorous acid, therefore after 2 months, a shocking agent like dichlor can regenerate the DMH and stop excess DMH from tying-up chlorine atoms. It should be noted that this excess DMH in the water is believed to also increase the solubility of the silver ions.

Reducing the concentration of the chlorine required to effectively sanitize the body of recreational water suitable for human partial immersion in the body of recreation while extending the use life of the body of recreational water before a required chlorine shocking of the recreational body of water provides the benefit of reduce damage to the bodies of recreational waters and the equipment associated therewith while also allowing the pool users to enjoy the beneficial sanitizing properties of chlorine.

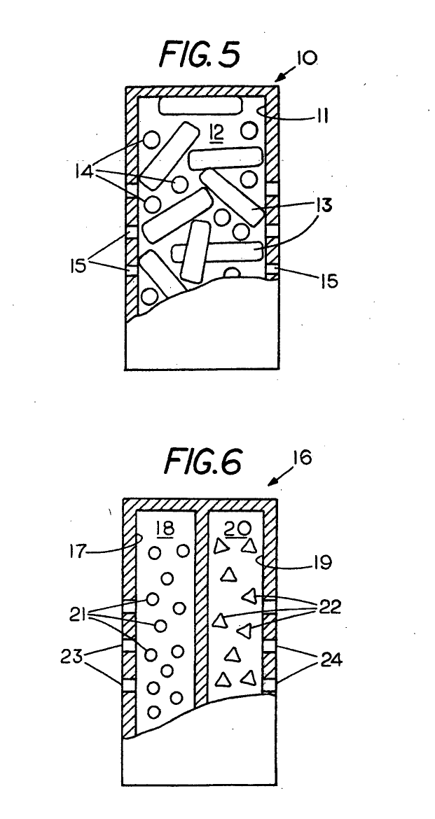

Referring to FIGS. 5 and 6, FIG. 5 shows an embodiment of an apparatus of the present invention comprising a dispenser 10 having a housing 11 containing a compartment 12 therein. Located in compartment 12 is a source of DCDMH 13 and a bactericide comprising a silver ion donor such as silver chloride 14. A set of openings 15 allows water access to compartment 12 and to the source of DCDMH 13 and the silver chloride 14.

FIG. 6 shows an alternative embodiment of an apparatus of the present invention comprising a dispenser 16 having a first housing 17 containing a compartment 18 and a second housing 19 with a compartment 20 therein. Located in compartment 18 is a silver ion donor such as silver chloride 21 and located in compartment 20 is a source of 1,3-Dichloro-5,5-dimethylhydantoin compound 22. A set of openings 23 allows water access to compartment 18 and to the silver chloride 21. Set of openings 23 may be fix or adjustable to allow for the releasing of a concentration of silver ions that lowers the concentration of chlorine required to effectively sanitize the body of recreational water when contacted by a body of recreational water containing a concentration of chlorine. Similarly, a set of openings 24 allows water access to compartment 20 and the source of 1,3-Dichloro-5,5-dimethylhydantoin compound 22. Similar to set of openings 23, set of openings 243 may be fix or adjustable to allow for the releasing of a concentration of 1,3-Dichloro-5,5-dimethylhydantoin effective at extending the use life of the body of recreational by at least 2 folds and preferably 4 folds before a required chlorine shocking of the body of recreational water when contacted by the body of recreational water containing the concentration of chlorine and the concentration of silver ions compared to if chlorine was used alone in sanitizing the body of recreational water.

Although FIGS. 5 and 6 shows the use of the silver ion donor as comprising silver chloride, other types of silver ion donors and other alternative bactericides whose solubility can be changed in the presence of 1,3-Dichloro-5,5-dimethylhydantoin compound can also be used such as silver bromide.

In regards to the source of 1,3-Dichloro-5,5-dimethylhydantoin compound 13, 22, FIG. 6 shows the source of 1,3-Dichloro-5,5-dimethylhydantoin compound 22 in particle form with the aforementioned particles having an initial size that is larger than the size of opening 23 to prevent the 1,3-Dichloro-5,5-dimethylhydantoin compound particles from escaping through opening 23. FIG. 5 shows the source of 1,3-Dichloro-5,5-dimethylhydantoin compound 13 in tablet form. Various types of material, including but not limited to microcrystalline cellulose (MCC), may be used as a binder in the formation of the 1,3-Dichloro-5,5-dimethylhydantoin compound tablets which are tabletized with the metal ion donor so that both the 1,3-Dichloro-5,5-dimethylhydantoin compound and the metal ion donor can be placed in the body of fluid to be treated.

The present invention includes the step of placing the dispenser 10, 16 containing both the source of 1,3-Dichloro-5,5-dimethylhydantoin compound 13, 22 and the silver chloride 14, 21 in the body of recreational water such as a body of water support in a spa, hot tub or swimming pool that is sanitized at least in part by chlorine and allowing water containing a concentration of chlorine to come into contact with the silver chloride 14, 21 which will release a concentration of silver ions that lowers the concentration of chlorine required to effectively sanitize the body of recreational water.

When water containing the concentration of chlorine and the concentration of silver ions comes into contact with the source of 1,3-Dichloro-5,5-dimethylhydantoin compound 13, 22, the source of 1,3-Dichloro-5,5-dimethylhydantoin compound 13, 22 will release a concentration of 1,3-Dichloro-5,5-dimethylhydantoin effective at interacting with the silver ions and chlorine to extend the use life of the body of recreational by at least 2 folds and preferably 4 folds before a required chlorine shocking of the body of recreational water compared to if chlorine was used alone in sanitizing the body of recreational water.

The present invention can also include a method of extending a use life of a body of recreational water while reducing a concentration of chlorine required to effectively sanitize the body of recreational water comprising the steps of: (1) adding a sanitizing concentration of total available chlorine to the body of recreation water that is suitable for human partial immersion in the body of recreation water; (2) adding a source of metal ions and a source of 1,3-Dichloro-5,5-dimethylhydanto into the body of recreational water to lower the concentration of chlorine required to effectively sanitize the body of recreational water; (3) maintaining a concentration of metal ions and a concentration of the source of 1,3-Dichloro-5,5-dimethylhydantoin effective at extending the use life of the body of recreational by at least 2 folds before a required chlorine shocking of the recreational body of water.

The above method may also include the steps of (4) maintaining a chorine concentration in the body of recreational water between 0.25 ppm and 0.5 ppm and a concentration of 1,3-Dichloro-5,5-dimethylhydantoin between 5-25 ppm; (5) adding to the body of recreational water an amount of 1,3-Dichloro-5,5-dimethylhydantoin to maintain a concentration of 1,3-Dichloro-5,5-dimethylhydantoin effective at extending the use life of the body of recreational water by at least 4 folds before a required chlorine-based shocking of the recreational body of water; (6) adding a granular source of metal ions and a granular source of 1,3-Dichloro-5,5-dimethylhydantoin to the body of recreational water to lower the concentration of chlorine required to effectively sanitize the body of recreational water; (7) adding a slow dissolving puck or tablets containing a source of metal ions and a source of 1,3-Dichloro-5,5-dimethylhydantoin to the body of recreational water to lower the concentration of chlorine required to effectively sanitize the body of recreational water; (8) adding a source of silver ions to the body of recreational water and maintaining a concentration of silver ions in the body of recreational water; (9) maintaining a silver ion concentration of 1-3 ppb in the body of recreational water; (10) adding a source of silver ions comprising silver chloride to the body of recreational water; (11) placing the source of metal ions and the source of 1,3-Dichloro-5,5-dimethylhydantoin in a dispenser and then placing the dispenser into the body of recreational water.

Thus the invention includes a method of maintaining spa water suitable for human immersion during a period of at least two months of bather use without having to shock the spa water to remove chloramines by adding 1,3-Dichloro-5,5-dimethylhydantoin, chlorine and source of silver ions in the spa water to lower a sanitizing concentration of the chlorine to between 0.25 and 1.5 ppm.

We claim:

1. A method of extending a use life of a body of recreational water while reducing a concentration of chlorine required to effectively sanitize the body of recreational water comprising the steps of:
adding a sanitizing concentration of total available chlorine to the body of recreation water that is suitable for human partial immersion in the body of recreation water;
adding a source of metal ions to the body of recreational water to lower the concentration of chlorine required to effectively sanitize the body of recreational water; and
adding to the body of recreational water an amount of 1,3-Dichloro-5,5-dimethylhydantoin to maintain a concentration of 1,3-Dichloro-5,5-dimethylhydantoin effective at extending the use life of the body of recreational by at least 2 folds before a required shocking of the recreational body of water while maintaining the biocidal effectiveness of the silver ions.

2. The method of claim 1 wherein the chorine concentration in the body of recreational water is maintained between 0.25 ppm and 0.5 ppm and the 1,3-Dichloro-5,5-dimethylhydantoin is maintained between 5-25 ppm.

3. The method of claim 1 wherein the step of adding 1,3-Dichloro-5,5-dimethylhydantoin to the body of recreational water comprising adding to the body of recreational water an amount of 1,3-Dichloro-5,5-dimethylhydantoin to maintain a concentration of 1,3-Dichloro-5,5-dimethylhydantoin effective at extending the use life of the body of recreational water at least 4 folds before a required chlorine-based shocking of the recreational body of water.

4. The method of claim 1 wherein the body of recreation water comprises a spa or hot tub.

5. The method of claim 1 wherein the 1,3-Dichloro-5,5-dimethylhydantoin is in either granular, puck or tablet form and the source of metal ions comprise a source of silver ions.

6. The method of claim 1 wherein 1,3-dichlor-5-ethyl-5-methylhydantoin is added to the body of water

7. The method of claim 1 including the step of maintaining the source of silver ion in the body of recreational water at a concentration of 1-3 ppb wherein the source of silver ions comprises silver chloride.

8. The method of claim 1 wherein the chlorine and the source of metal ions are the sole sanitizers in the body of recreational water.

9. The method of claim 1 wherein the steps of adding the source of metal ions to the body of recreational water and adding the amount of 1,3-Dichloro-5,5-dimethylhydantoin to the body of recreational water comprises placing the source of metal ions and the source of 1,3-Dichloro-5,5-dimethylhydantoin in a dispenser before placing the source of metal ions and the source of 1,3-Dichloro-5,5-dimethylhydantoin into the body of recreational water.

10. A method of maintaining spa water suitable for human immersion during a period of at least two months without having to shock the spa water to remove chloramines comprising the steps of:
adding chlorine and 1,3-Dichloro-5,5-dimethylhydantoin to the spa water;
placing a source of silver ions in the spa water to lower a sanitizing concentration of chlorine to between 0.25 and 1.5 ppm; and
subjecting the spa water to bather use while maintaining the sanitizing concentration of chlorine.

11. The method of claim 10 including maintaining the sanitizing concentration of chorine in the spa water between 0.25 ppm and 0.5 ppm and the 1,3-Dichloro-5,5-dimethylhydantoin between 5-25 ppm by periodically adding 1,3-Dichloro-5,5-dimethylhydantoin to a dispenser.

12. The method of claim 10 including the step of adding 3-Dichloro-5,5dimethylhydantoin; 1,3-Dichloro-5-ethyl-5-methylhydantoin and monochloro-5-methylhydantoin in combination with the 1,3-Dichloro-5,5-dimethylhydantoin.

13. A method of extending a use life of a body of recreational water while reducing a concentration of chlorine required to effectively sanitize the body of recreational water comprising the steps of:
adding a sanitizing concentration of total available chlorine to the body of recreation water that is suitable for human partial immersion in the body of recreation water;
adding a source of silver ions and a source of 1,3-Dichloro-5,5-dimethylhydantoin to the body of recreational water to lower the concentration of chlorine required to effectively sanitize the body of recreational water; and
maintaining a concentration of silver ions and a concentration of the source of 1,3-Dichloro-5,5-dimethylhydantoin effective at extending the use life of the body of recreational by at least 2 folds before a required chlorine shocking of the recreational body of water.

14. The method of claim 13 wherein the chorine concentration in the body of recreational water is maintained between 0.25 ppm and 0.5 ppm and the 1,3-Dichloro-5,5-dimethylhydantoin is maintained between 5-25 ppm.

15. The method of claim 14 wherein the source of silver ions comprises silver chloride and the 1,3-Dichloro-5,5-dimethylhydantoin is in granular form.

16. The method of claim 14 including the step of maintaining the silver ion in the body of recreational water at a concentration of 1-3 ppb.

17. The method of claim 14 wherein the steps of adding the source of silver ions to the body of recreational water and adding the amount of 1,3-Dichloro-5,5-dimethylhydantoin to the body of recreational water comprises placing the source of silver ions and the source of 1,3-Dichloro-5,5-dimethylhydantoin in a dispenser and then placing dispenser into the body of recreational water.

18. The method of claim 14 wherein the steps of maintaining a concentration of the source of silver ions and a concentration of the source of 1,3-Dichloro-5,5-dimethylhydantoin comprises maintaining a concentration of the source of silver ions and a concentration of the source of 1,3-Dichloro-5,5-dimethylhydantoin effective at extending the use life of the body of recreational by at least 4 folds before a required chlorine shocking of the recreational body of water.

19. A dispenser for extending a use life of a body of recreational water sanitized at least in part by chlorine while reducing a concentration of chlorine required to effectively sanitize the body of recreational water comprising:
a first housing having a water accessible compartment containing a silver ion donor, the water accessible compartment of the first housing releasing a concentration of silver ions that lowers the concentration of chlorine required to effectively sanitize the body of recreational water when contacted by the body of recreational water containing a concentration of chlorine;
a second housing having a water accessible compartment containing a source of 1,3-Dichloro-5,5-dimethylhydantoin, the water accessible compartment of the second housing releasing a concentration of 1,3-Dichloro-5,5-dimethylhydantoin effective at extending the use life of the body of recreational by at least 2 folds before a required chlorine shocking of the body of recreational water when contacted by the body of recreational water containing the concentration of chlorine and the concentration of silver ions.

20. The dispenser of claim 19 wherein the silver ion donor comprises silver chloride,
